(19) United States
(12) Patent Application Publication
(10) Pub. No.: US 2024/0197427 A1
(43) Pub. Date: Jun. 20, 2024

(54) Collapsible Lateral Table Drape for Surgical Equipment

(71) Applicant: O&M Halyard, Inc., Mechanicsville, VA (US)

(72) Inventors: Marshall R. Dean, Cumming, GA (US); Namita A. Mithani, Alpharetta, GA (US); Malak Bayyari, Fayetteville, AR (US)

(21) Appl. No.: 18/537,907

(22) Filed: Dec. 13, 2023

Related U.S. Application Data
(60) Provisional application No. 63/432,579, filed on Dec. 14, 2022.

(51) Int. Cl. A61B 46/10; A61B 46/00

(52) U.S. Cl. CPC A61B 46/10; A61B 46/40; A61B 2090/3764

(57) ABSTRACT

A drape for providing a sterile field around a portion of an imaging device is provided. The drape includes an upper panel having an upper surface and a lower surface; a front panel having an outer surface and an inner surface; and a rear panel having an outer surface and an inner surface. Further, a side edge of the rear panel and a side edge of the front panel define an opening therebetween.

### RELATED APPLICATIONS

The present application claims priority to U.S. Provisional Patent Application No. 63/432,579, having a filing date of Dec. 14, 2022, the entire contents of which are incorporated herein by references.

### FIELD

The present invention relates to medical drapes used to cover surgical equipment such as imaging devices during surgical procedures or other medical procedures where maintaining a sterile field is required.

### BACKGROUND

During surgical procedures in the operating room, there is often a need for intraoperation x-rays to be taken. In such instances, a piece of equipment commonly referred to as a “C-Arm” due to its C-shape is used to position an x-ray emitter and receiver of an imaging device around the patient and surgical field. The C-Arm extends nearly fully around the table, including underneath, which is an explicitly non-sterile zone. Thus, this creates a need for a drape that extends the horizontal sterile surgical field to create a barrier between the C-Arm and the sterile field in which the surgical procedure is taking place. Because of the large size and arcuate shape of a C-arm imaging device, attempts have been made to cover the C-arm imaging device with multiple sheets of surgical drapes to maintain the sterile field. However, the use of such multiple sheets can become cumbersome.

Therefore, a need exists for a single surgical drape that can be used to cover the C-arm portion of the imaging device to maintain a sterile field.

### SUMMARY

Objects and advantages of the present disclosure will be set forth in part in the following description, or may be obvious from the description, or may be learned through practice of the present disclosure.

In one embodiment, the present disclosure is directed to a drape for providing a sterile field around a portion of an imaging device. The drape includes an upper panel having an upper surface and a lower surface; a front panel having an outer surface and an inner surface; and a rear panel having an outer surface and an inner surface. Further, a side edge of the rear panel and a side edge of the front panel define an opening therebetween.

In one embodiment, the rear panel and the upper panel can each have a rectangular or linear shape.

In another embodiment, the rear panel can have a curvilinear shape and the upper panel can have a rectangular or linear shape.

In another embodiment, the front panel has a rectangular or linear shape.

In another embodiment, the rear panel and the upper panel can each include a curved section and a linear section extending from an end point of the curved section towards the front panel.

In addition, an angle α formed between an edge defined by the linear section and an axis extending from the end point of the curved section and intersecting with the front edge of the upper panel to form a 90 degree angle therewith can range from about 5 degrees to about 45 degrees as measured from the axis.

In yet another embodiment, attachment means can be positioned on the upper surface of the upper panel adjacent an upper edge of the front panel, and wherein corresponding attachment means can be positioned on the upper surface of the upper panel adjacent an upper edge of the rear panel.

Further, the attachment means on the upper surface of the upper panel adjacent the upper edge of the front panel can be aligned with the corresponding attachment means on the upper surface of the upper panel adjacent an upper edge of the rear panel when the drape is folded inwardly along one or more fold lines to maintain the sterility of the upper panel prior to use of the drape in a surgical procedure.

In an additional embodiment, the upper panel can include one or more pleats, wherein the pleats are expandable outwardly when the drape is in use to surround the portion of the imaging device.

In one more embodiment, at least one handle can be present on the rear panel.

In another embodiment, a support structure can be attached at an upper edge of the rear panel, a lower edge of the rear panel, or a combination thereof.

In still another embodiment, an attachment means can be disposed on the lower surface of the upper panel adjacent the front panel.

In yet another embodiment, the front panel can be positioned a distance away from a front edge of the upper panel toward the rear panel.

In one more embodiment, the imaging device can be a C-arm imaging device.

In an additional embodiment, the drape can be free of sidewalls positioned between the front panel and the rear panel.

In one more embodiment, a first side edge of the rear panel and a first side edge of the front panel can define the opening, further wherein a sidewall is positioned between a second side edge of the rear panel and a second side edge of the front panel.

In another embodiment, the upper panel, the front panel, the rear panel, or a combination thereof can be translucent, transparent, and/or radiolucent.

In still another embodiment, the upper panel, the front panel, the rear panel, or a combination thereof can be formed from a nonwoven material.

In yet another embodiment, the nonwoven material can include a spunbond material, a meltblown material, or a combination thereof.

In an additional embodiment, the upper panel, the front panel, the rear panel, or a combination thereof can be formed from a film. Further, the film can include polyethylene.

In one more embodiment, the drape can be sterilizable.

These and other features, aspects, and advantages of the present disclosure will become better understood with reference to the following description and appended claims. The accompanying drawings, which are incorporated in and constitute a part of this specification, illustrate embodiments of the present disclosure and, together with the description, serve to explain the principles of the present disclosure.

### BRIEF DESCRIPTION OF THE FIGURES

A full and enabling disclosure of the present disclosure, including the best mode thereof, directed to one of ordinary skill in the art, is set forth in the specification, which makes reference to the appended figures, in which:

FIG. 1 illustrates a top perspective view of one embodiment of the drape of the present disclosure;

FIG. 2 illustrates a bottom perspective view of one embodiment of the drape of the present disclosure;

FIG. 3 illustrates a top view of one embodiment of the drape of the present disclosure;

FIG. 4 illustrates a top perspective view of another embodiment of the drape of the present disclosure;

FIG. 5 illustrates a bottom perspective view of another embodiment of the drape of the present disclosure;

FIG. 6 illustrates a top view of another embodiment of the drape of the present disclosure;

FIG. 7 illustrates a cross-section of the drape of the drapes of the present disclosure in a collapsed or folded state;

FIG. 8 illustrates a top perspective view of one embodiment of the drape of the present disclosure;

FIG. 9 illustrates a top perspective view of another embodiment of the drape of the present disclosure;

FIG. 10 illustrates a top perspective view of yet another embodiment of the drape of the present disclosure;

FIG. 11A illustrates a perspective view of an embodiment of the drape of the present disclosure when attached to an operating table and used to cover a C-arm portion of imaging equipment when the pleats have not been expanded; and

FIG. 11B illustrates a perspective view of an embodiment of the drape of the present disclosure when attached to an operating table and used to cover a C-arm portion of imaging equipment when the pleats have been expanded.

### DETAILED DESCRIPTION

Reference will now be made in detail to one or more embodiments of the present disclosure, examples of the present disclosure, examples of which are illustrated in the drawings. Each example and embodiment is provided by way of explanation of the disclosure and is not meant as a limitation of the disclosure. For example, features illustrated or described as part of one embodiment may be used with another embodiment to yield still a further embodiment. It is intended that the disclosure include these and other modifications and variations as coming within the scope and spirit of the disclosure.

As used herein, the terms “about,” “approximately,” or “generally,” when used to modify a value, indicates that the value can be raised or lowered by 5% and remain within the disclosed embodiment. Further, when a plurality of ranges are provided, any combination of a minimum value and a maximum value described in the plurality of ranges are contemplated by the present disclosure. For example, if ranges of “from about 20% to about 80%” and “from about 30% to about 70%” are described, a range of “from about 20% to about 70%” or a range of “from about 30% to about 80%” are also contemplated by the present disclosure.

Generally speaking, the present disclosure is directed to a drape for providing a sterile field around a portion of an imaging device. The drape includes an upper panel having an upper surface and a lower surface; a front panel having an outer surface and an inner surface; and a rear panel having an outer surface and an inner surface. Further, at least one side edge of the rear panel and at least one side edge of the front panel define an opening therebetween, and it is to be understood that both side edges of the rear panel and both side edges of the front panel may define an opening therebetween. In other words, the drape is free of at least one sidewall and possibly two sidewalls. The present inventors have found that the opening between the rear panel and the front panel allows for efficient placement of the drape over the desired portion of an imaging device, such as a C-arm, while not jeopardizing the ability of the drape to provide a sterile field that can extend the horizontal sterile field during a surgical procedure to create a barrier between the C-arm and the operating area. The material from which the drape is formed can include a film, a nonwoven material, or a combination thereof. For instance, the film can be a polyethylene film such as a low density polyethylene (LDPE), medium density polyethylene, or a high density polyethylene film (HDPE). Further, the nonwoven material can be a spunbond material, a meltblown material, or a combination thereof. For example, the nonwoven material can be a spunbond-meltblown-spunbond (SMS) laminate. Additionally, all though not required, in some embodiments, the drape material can be translucent or transparent such that the imaging device which it is used to cover can be seen through the drape material to aid a medical professionally in accurately positioning the imaging device to take images of a specific area of interest on a patient.

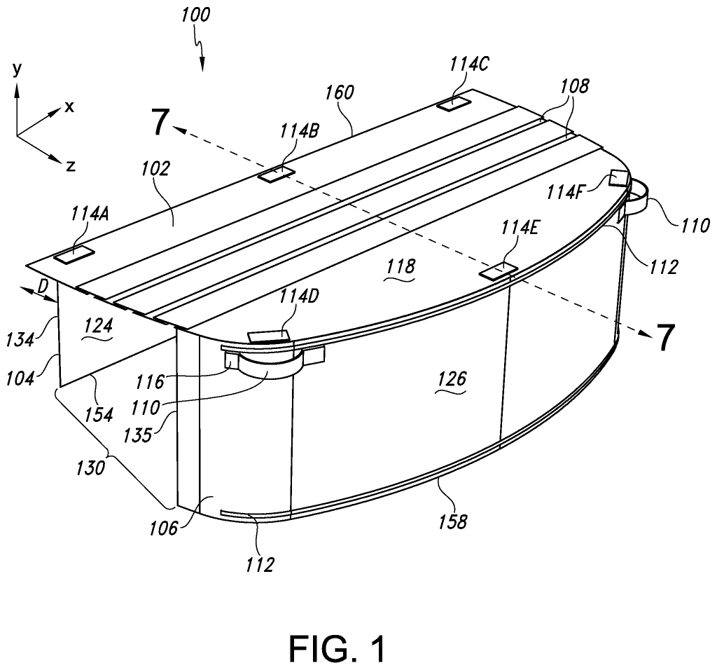

Referring now to FIGS. 1-11B, the various features of the device contemplated by the present disclosure will be described in more detail.

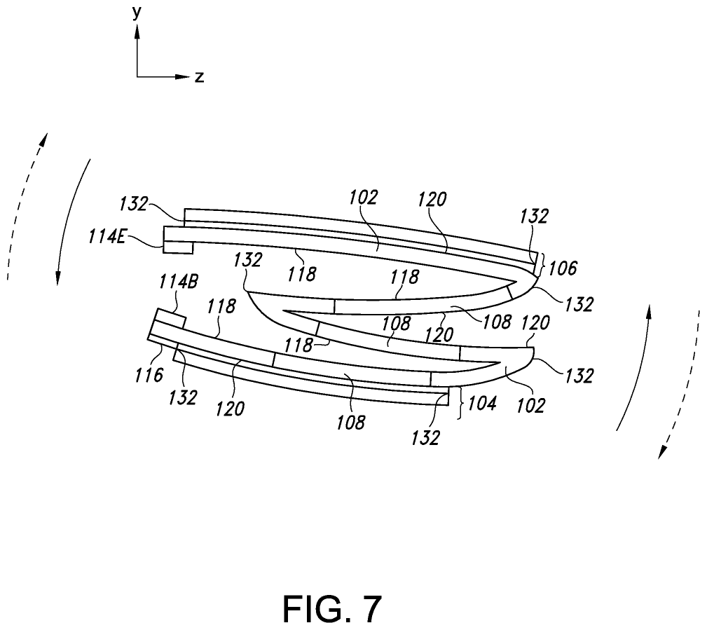

Turning first to FIG. 1, a top perspective view of one embodiment of a drape 100 is shown. The drape 100 can be a lateral table drape that can be attached to, for example, an operating table during a medical procedure. The drape 100 has an upper panel 102, a front panel 104, and a rear panel 106. The front panel 104 and the rear panel 106 can extend horizontally along an x-axis and vertically along a y-axis and can be attached to the upper panel 102. In addition, the front panel 104 can have side edges 134 that extend vertically along the y-axis, and the rear panel 106 can have side edges 135 that also extend vertically along the y-axis. The front panel 104 also has an outer surface 122, an inner surface 124, an upper edge 152, and a lower edge 154. Likewise, the rear panel 106 has an outer surface 126, and inner surface 128, an upper edge 156, and a lower edge 158. Meanwhile, the upper panel 102 can extend laterally along a z-axis between the front panel 104 and the rear panel 106. As shown, the upper panel 102 can include first attachment means 114A, 114B, 114C, 114D, 114E, and 114F positioned about the periphery of the upper panel 102. For example, first attachment means 114A, 114B, and 114C can be positioned along a front edge 160 of the upper panel 102, while first attachment means 114D, 114E, and 114F can be positioned along a rear edge 162 of the upper panel 102. The first attachment means 114A, 114B, 114C, 114D, 114E, and 114F can be in the form of hook and loop fasteners, a tape with a removable release liner, an adhesive with a release liner, or any other suitable attachment means. The manner in which the first attachment means 114A, 114B, 114C, 114D, 114E, and 114F to help maintain the sterility of the drape 100 are utilized is discussed in more detail below with respect to FIG. 7.

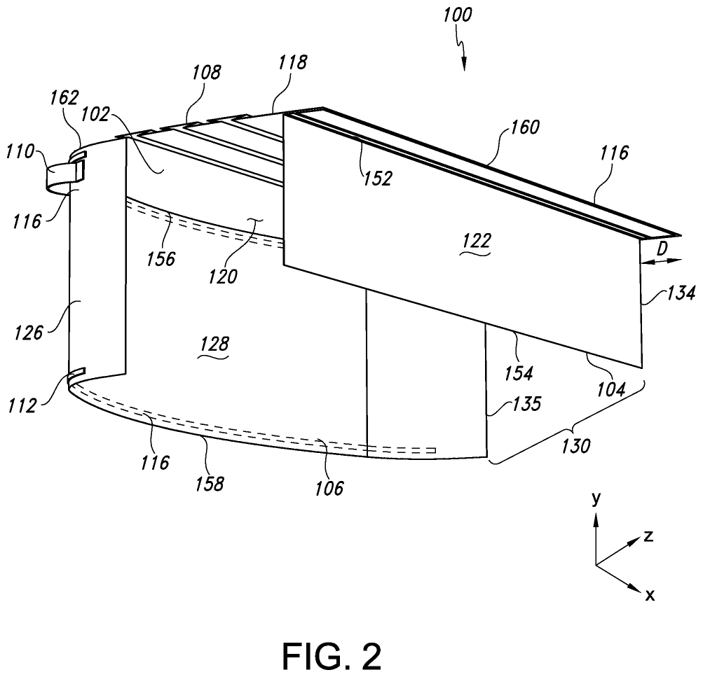

Also, as shown in FIGS. 1 and 2, the upper panel 102 can include one or more pleats 108 formed from the drape material that can expand in an accordion-like fashion when the drape 100 is used to cover a portion of an imaging device. The pleats 108 also define fold lines 132 (see FIG. 7) where the drape 100 can be folded, such as in a z-shape) so that the upper panel 102 can be collapsed or folded on itself and secured with attachment means 114A, 114B, 114C, 114D, 114E, and 114F to protect the sterility of the upper panel 102 until in use during a medical procedure. Further, the drape 100 can be returned to the collapsed or folded state to preserve the sterility of the upper panel 102 if the imaging equipment needs to be moved, after which the drape 100 can be unfolded to maintain a sterile field after the imaging equipment is moved to a different location or position.

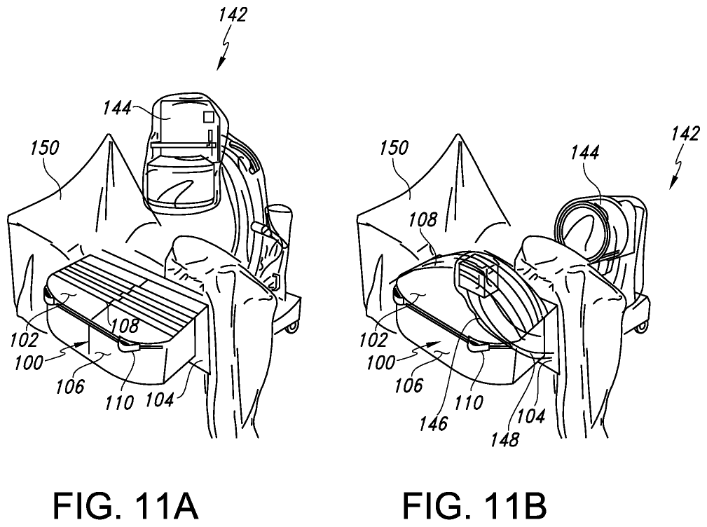

In addition, a support structure such as wire 112, which can be a coated wire, can be attached at the upper edge 156 of the rear panel 102, a lower edge 158 of the rear panel 102, or a combination thereof. The wire 112 can provide structure and support to the drape 100 to position the drape around the portion of an imaging device 142 to be covered (see FIGS. 11A and 11B, discussed in more detail below). The wire 112 can be attached to the rear panel 102 via second attachment means 116, such as an adhesive, double-side tape, or any other suitable attachment means. Further, it should be understood that any material that is flexible that can also provide rigidity and support to the drape 100 can be utilized instead of the coated wire, such as a flexible plastic or metal. Additionally, the front panel 102 can have at least one handle 110 located near an upper edge 156 of the rear panel 102 and on its outer surface 126. The at least one handle 110 can allow a user to properly position the drape 100 around the medical equipment (e.g., imaging device) to be covered.

Moreover, a gap or opening 130 can be defined between each of the side edges 134 of the front panel 104 and each of the side edges 135 of the rear panel 106 such that the rear panel 106 is free of sidewalls and does not contact the edges 134 of the front panel 104. The opening 130 can allow for more efficient and less bulky covering of any medical equipment underneath the drape 100 and allows for easier manipulation of the drape 100 around the medical equipment.

Further, referring to FIG. 2, which shows a bottom perspective view of the drape 100 of FIG. 1, the upper panel 102 can have an upper surface 118 and a lower surface 120. In addition, the upper panel 102 can have a front edge 160 that is located a distance D along the z-axis beyond the upper edge 152 of the front panel 104. In other words, the front panel 104 can be positioned a distance D away from the front edge 160 of the upper panel 102 toward the rear panel 106 along the z-axis. The front panel 104 can also define a rear edge 162. Moreover, second attachment means 116, such as an adhesive, double-side tape, or any other suitable attachment means, can be disposed on the lower surface 120 of the upper panel 102 adjacent the front panel 104 between a front edge 160 of the upper panel 102 and an upper edge 142 of the front panel 104, where such attachment means 116 can be utilized to attach the upper panel 102 of the drape 100 to a patient drape 150 (see FIGS. 11A and 11B), an operating table, or any other object around which a sterile field should be maintained.

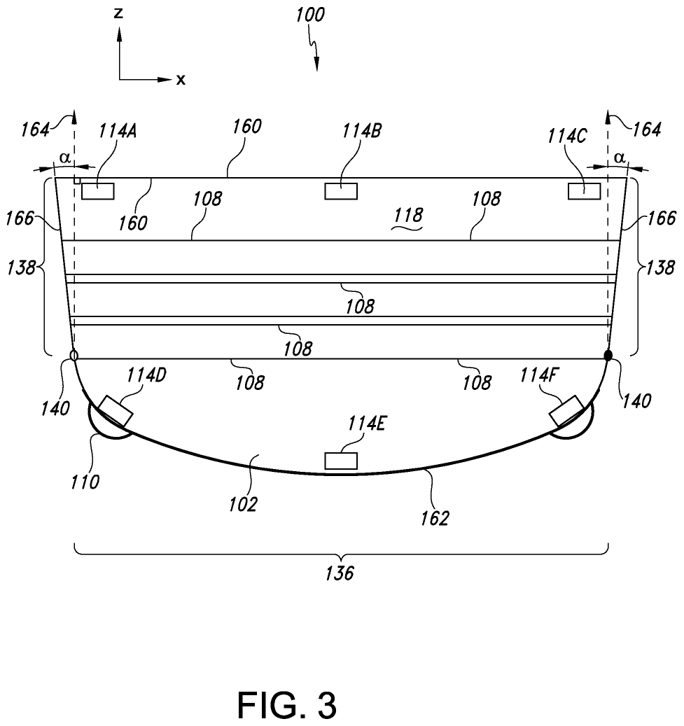

In addition, FIG. 3 illustrates a top view of one embodiment of the drape 100 showing in detail a curved section 136 of the upper panel 102 and a linear section 138 that extends from an end point 140 of the curved section 136. In addition, an angle α formed between an edge 166 defined by the linear section 138 and an axis 164 extending from the end point 140 of the curved section 136 and intersecting with the front edge 160 of upper panel 102 to form a 90 degree angle 164 therewith can range from about 5 degrees to about 45 degrees, such as from about 7.5 degrees to about 30 degrees, such as from about 10 degrees to about 25 degrees, as measured from the axis 164. Further, referring back to FIG. 2, it should be noted that the rear panel 104 can have a shape that corresponds with the curved section 136 of the upper panel 102 and can be attached to the curved section 136 by any suitable means, including, but not limited to, heat sealing, adhesive, double bonded tape, ultrasonic bonding, etc. Meanwhile, the front panel 104 can have a shape that corresponds with a shape of the front edge 160 of the upper panel 102 (e.g., a linear or straight section). Further, although not required, the upper edge 152 of the front panel 104 can have a length along the x-axis that the same as a length of the front edge of the upper panel 102 along the x-axis (see FIG. 2). However, in other embodiments, the upper edge 152 of the front panel 104 can have a length along the x-axis that is less than a length of the front edge of the upper panel 102 along the x-axis.

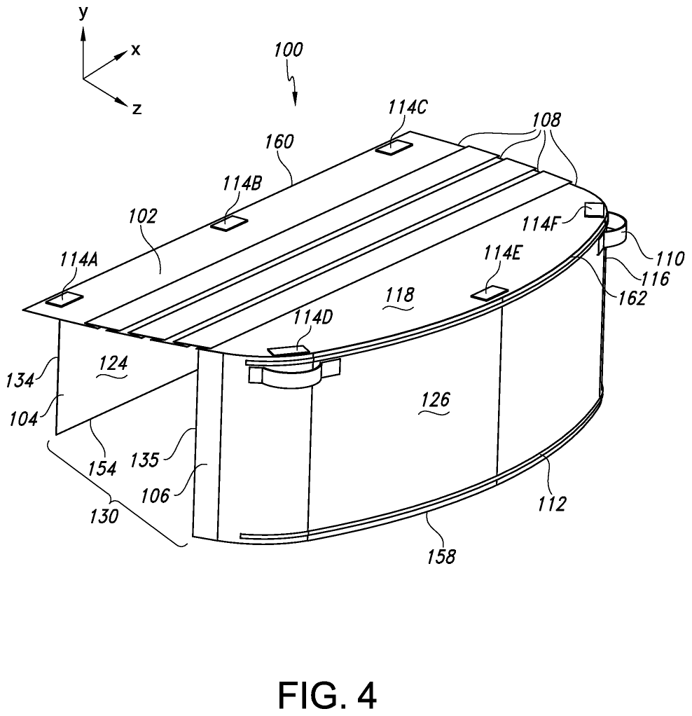

Turning now to FIG. 4, a top perspective view of another embodiment of a drape 100 contemplated by the present disclosure is shown. As with the drape of FIG. 1, the drape 100 can be a lateral table drape that can be attached to, for example, an operating table during a medical procedure. The drape 100 has an upper panel 102, a front panel 104, and a rear panel 106. The front panel 104 and the rear panel 106 can extend horizontally along an x-axis and vertically along a y-axis and can be attached to the upper panel 102. In addition, the front panel 104 can have side edges 134 that extend vertically along the y-axis, and the rear panel 106 can have side edges 135 that also extend vertically along the y-axis. The front panel 104 also has an outer surface 122, an inner surface 124, an upper edge 152, and a lower edge 154. Likewise, the rear panel 106 has an outer surface 126, and inner surface 128, an upper edge 156, and a lower edge 158. Meanwhile, the upper panel 102 can extend laterally along a z-axis between the front panel 104 and the rear panel 106. As shown, the upper panel 102 can include first attachment means 114A, 114B, 114C, 114D, 114E, and 114F positioned about the periphery of the upper panel 102. For example, first attachment means 114A, 114B, and 114C can be positioned along a front edge 160 of the upper panel 102, while first attachment means 114D, 114E, and 114F can be positioned along a rear edge 162 of the upper panel 102. The first attachment means 114A, 114B, 114C, 114D, 114E, and 114F can be in the form of hook and loop fasteners, a tape with a removable release liner, an adhesive with a release liner, or any other suitable attachment means. The manner in which the first attachment means 114A, 114B, 114C, 114D, 114E, and 114F to help maintain the sterility of the drape 100 are utilized is discussed in more detail below with respect to FIG. 7.

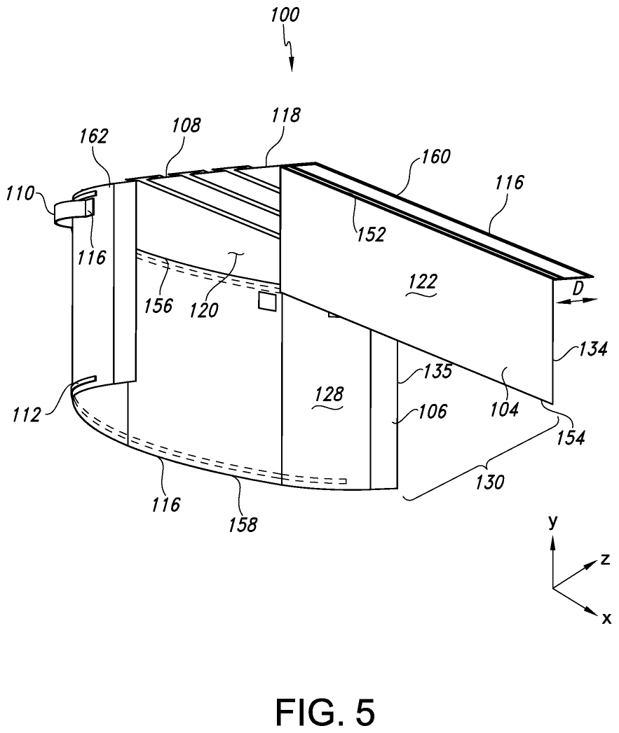

Also, as shown in FIGS. 4 and 5, the upper panel 102 can include one or more pleats 108 formed from the drape material that can expand in an accordion-like fashion when the drape 100 is used to cover a portion of an imaging device. The pleats 108 also define fold lines 132 (see FIG. 7) where the drape 100 can be folded, such as in a z-shape) so that the upper panel 102 can be collapsed or folded on itself and secured with attachment means 114A, 114B, 114C, 114D, 114E, and 114F to protect the sterility of the upper panel 102 until in use during a medical procedure. Further, the drape 100 can be returned to the collapsed or folded state to preserve the sterility of the upper panel 102 if the imaging equipment needs to be moved, after which the drape 100 can be unfolded to maintain a sterile field after the imaging equipment is moved to a different location or position.

In addition, a support structure, such as a wire 112, which can be a coated wire, can be attached at the upper edge 156 of the rear panel 102, a lower edge 158 of the rear panel 102, or a combination thereof. The wire 112 can provide structure and support to the drape 100 to position the drape around the portion of an imaging device 142 to be covered (see FIGS. 11A and 11B, discussed in more detail below). The wire 112 can be attached to the rear panel 102 via second attachment means 116, such as an adhesive, double-side tape, or any other suitable attachment means. Further, it should be understood that any material that is flexible that can also provide rigidity and support to the drape 100 can be utilized instead of the coated wire, such as a flexible plastic or metal. Additionally, the front panel 102 can have at least one handle 110 located near an upper edge 156 of the rear panel 102 and on its outer surface 126. The at least one handle 110 can allow a user to properly position the drape 100 around the medical equipment (e.g., imaging device) to be covered.

Moreover, a gap or opening 130 can be defined between each of the side edges 134 of the front panel 104 and each of the side edges 135 of the rear panel 106 such that the rear panel 106 is free of sidewalls and does not contact the edges 134 of the front panel 104. The opening 130 can allow for more efficient and less bulky covering of any medical equipment underneath the drape 100 and allows for easier manipulation of the drape 100 around the medical equipment.

Further, referring to FIG. 5, which shows a bottom perspective view of the drape 100 of FIG. 4, the upper panel 102 can have an upper surface 118 and a lower surface 120. In addition, the upper panel 102 can have a front edge 160 that is located a distance D along the z-axis beyond the upper edge 152 of the front panel 104. In other words, the front panel 104 can be positioned a distance D away from the front edge 160 of the upper panel 102 toward the rear panel 106 along the z-axis. The front panel 104 can also define a rear edge 162. Moreover, second attachment means 116, such as an adhesive, double-side tape, or any other suitable attachment means, can be disposed on the lower surface 120 of the upper panel 102 adjacent the front panel 104 between a front edge 160 of the upper panel 102 and an upper edge 142 of the front panel 104, where such attachment means 116 can be utilized to attach the upper panel 102 of the drape 100 to a patient drape 150 (see FIGS. 11A and 11B), an operating table, or any other object around which a sterile field should be maintained.

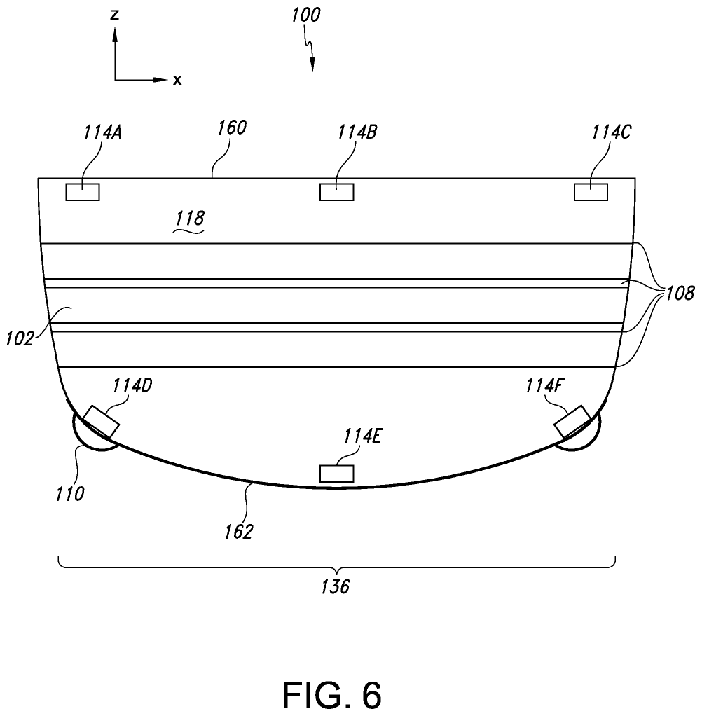

In addition, FIG. 6 illustrates a top view of one embodiment of the drape 100 showing in detail a curved section 136 that extends from one end of the upper edge 142 of the front panel 104 to the other end of the upper edge 142 of the front panel in that it does not have a linear section 138 as shown in FIG. 3. Further, referring back to FIG. 5, it should be noted that the rear panel 104 can have a shape that corresponds with the curved section 136 of the upper panel 102 and can be attached to the curved section 136 by any suitable means, including, but not limited to, heat sealing, adhesive, double bonded tape, ultrasonic bonding, etc. Meanwhile, the front panel 104 can have a shape that corresponds with a shape of the front edge 160 of the upper panel 102 (e.g., a linear or straight section). Further, although not required, the upper edge 152 of the front panel 104 can have a length along the x-axis that the same as a length of the front edge of the upper panel 102 along the x-axis (see FIG. 5). However, in other embodiments, the upper edge 152 of the front panel 104 can have a length along the x-axis that is less than a length of the front edge of the upper panel 102 along the x-axis.

Turning now to FIG. 7, the manner in which the drape 100 (or drapes 200, 300, or 400 described below) can be collapsed or folded to maintain the sterility of the upper panel 102 is shown. FIG. 7 is a cross-sectional view of FIG. 1 taken at line 7-7 and shows how the drape 100 can be folded or collapsed (solid arrow) or unfolded (dashed arrow). When the drape 100 is folded or collapsed on itself to maintain the sterility of the upper surface 120 of the upper panel 102, attachment means 114B can be joined to 114E as shown, and attachment means 114A can be joined to 114D and attachment means 114C and be joined to 114F in a similar manner. In order to collapse the drape 100, the drape 100 can be folded along fold lines 132 in between pleats 108 to form a z-like shape. In this manner, when collapsed, the upper surface 120 remains contained within the other panels of the drape 100 such that sterility is maintained. Further, in addition to the upper panel 102 being folded, the front panel 104 and the rear panel 106 can also be folded along fold lines 132 for packaging and sterilization (e.g., via ethylene oxide, hydrogen peroxide vapor, steam, etc.).

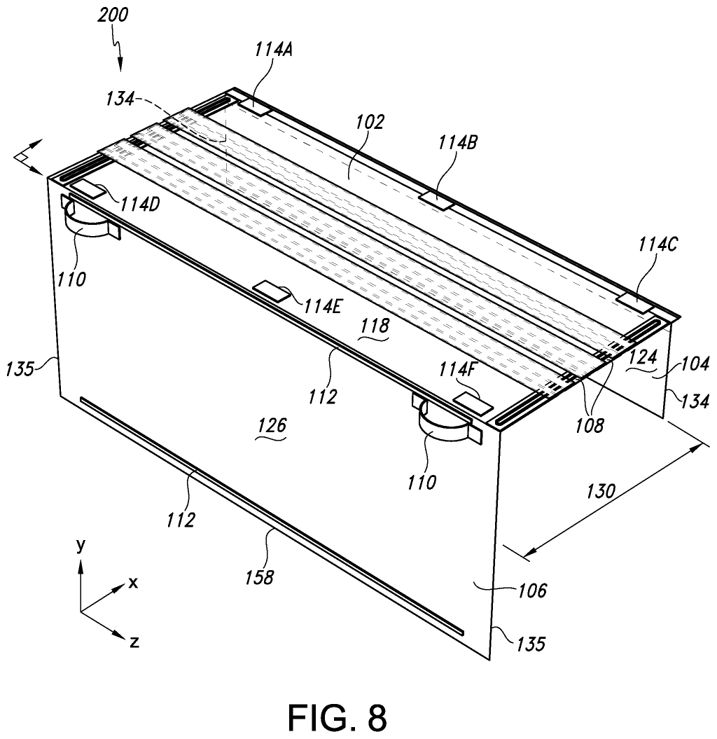

Turning now to FIG. 8 illustrations an additional embodiment of a drape 200 contemplated by the present disclosure. Although similar to drape 100 in that the drape 200 includes a gap or opening 130 that is defined between each of the side edges 134 of the front panel 104 and each of the side edges 135 of the rear panel 106 such that the rear panel 106 is free of sidewalls and does not contact the edges 134 of the front panel 104, the drape 200 of FIG. 8 does not include an upper panel 102 or rear panel 106 having a curved section 136. Instead, the upper panel 102 is generally rectangular and/or linear such that the angle at which the upper panel 102 meets the side edges 134 of the front panel 104 and the side edges 135 is generally a right angle or 90 degrees. Additionally, the rear panel 106 is also generally linear and rectangular.

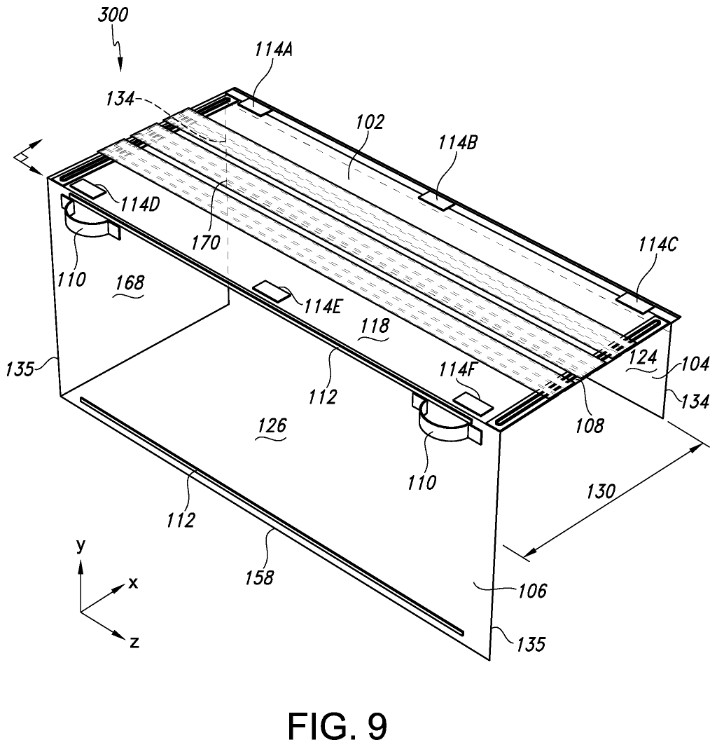

Further, FIG. 9 illustrates another embodiment of a drape 300 contemplated by the present disclosure. On one side, the drape 300 includes one gap or opening 130 that is defined between one of the side edges 134 of the front panel 104 and one of the side edges 135 of the rear panel 106. Meanwhile, on the other side of the drape material 300, there is no such gap or opening between the side edge 134 and the side edge 135. In other words, on one side of the drape 300, there is a sidewall 168, where respective edges of the sidewall 170 contact one side edge 134 of the front panel 104 and one side edge 135 of the rear panel 106, where the sidewall 168 also contacts the upper panel 102 at its edge. Additionally, the drape 300 of FIG. 9 does not include an upper panel 102 having a curved section 136. Instead, the upper panel 102 is generally rectangular and/or linear such that the angle at which the upper panel 102 meets the side edges 134 of the front panel 104 and the side edges 135 is generally a right angle or 90 degrees.

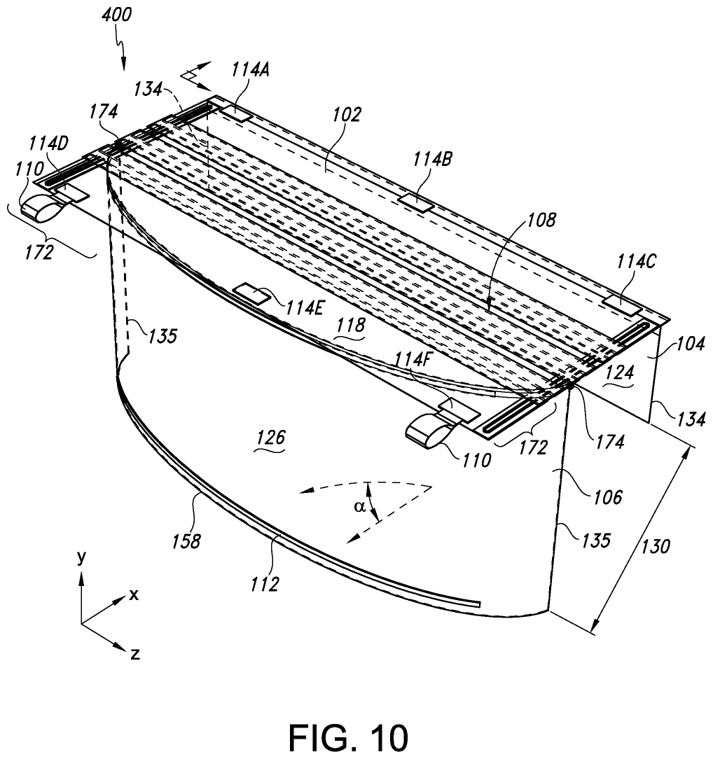

In addition, FIG. 10 illustrates one more embodiment of a drape 400 contemplated by the present disclosure. While similar to the drape 200 of FIG. 8 in terms of the shape of the upper panel 102 and the lack of sidewalls between the front panel 104 and the rear panel 106 due to the gaps 130 defined between respective side edges 134 and 135 of the front panel 104 and rear panel 106, the rear panel 106 has a curvilinear shape while the upper panel 102 has a linear or rectangular shape such that the side edges 135 of the rear panel 106 meet or contact the upper panel 102 at a point 174, creating an overlap 172 of the upper panel 102 beyond the rear panel 106 in the x-direction. This overlap defines an angle α with respect to the x-axis that can range from about 10 degrees to about 60 degrees, such as from about 15 degrees to about 60 degrees, such as from about 20 degrees to about 40 degrees. Without intending to be limited by any particular theory, the present inventors have found that the curvilinear nature of the rear panel 106 can provide for additional coverage of medical equipment.

Next, FIG. 11A illustrates a perspective view of an embodiment of the drape 100 (or drapes 200, 300, or 400) of the present disclosure when attached to a patient drape 150 an operating table. In FIG. 11A, an imaging device 142 is shown having an upper imaging section 144 and a lower imaging section 146 that includes a C-arm portion 148 (not shown, see FIG. 11B). The lower imaging section 146 has not yet been moved under the patient drape 150 and table such that the pleats 108 on the drape 100 of the present disclosure have not been expanded. Meanwhile, FIG. 11B illustrates a perspective view of an embodiment of the drape 100 of the present disclosure when attached to the patient drape 150 after the C-arm portion 148 of the imaging device 142 has been moved under the patient drape 150 and operating table. The pleats 108 on the upper panel 102 have expanded to cover the C-arm portion 148 and provide a sterile field around the lower imaging section 146 of the imaging device 142.

While the present disclosure has been described with reference to certain exemplary embodiments thereof, those skilled in the art may make various modifications to the described embodiments of the present disclosure without departing from the scope of the disclosure. The terms and descriptions used herein are set forth by way of illustration only and not meant as limitations. In particular, although the present disclosure has been described by way of examples, a variety of compositions and processes would practice the inventive concepts described herein. Although the disclosure has been described and disclosed in various terms and certain embodiments, the scope of the disclosure is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the disclosure as defined in the following claims and their equivalents.

What is claimed is:

1. A drape for providing a sterile field around a portion of an imaging device, the drape comprising:
an upper panel having an upper surface and a lower surface;
a front panel having an outer surface and an inner surface; and
a rear panel having an outer surface and an inner surface, wherein a side edge of the rear panel and a side edge of the front panel define an opening therebetween.

2. The drape of claim 1, wherein the rear panel has a rectangular shape and the upper panel has a rectangular shape.

3. The drape of claim 1, wherein the rear panel has a curvilinear shape and the upper panel has a rectangular shape.

4. The drape of claim 1, wherein the front panel has a rectangular shape.

5. The drape of claim 1, wherein the rear panel and the upper panel each include a linear section extending from an end point of a curved section towards the front panel, wherein an angle formed between an edge defined by the linear section and an axis extending from the end point of the curved section and intersecting with a front edge of the upper panel to form a 90 degree angle therewith ranges from about 5 degrees to about 45 degrees as measured from the axis.

6. The drape of claim 1, wherein attachment means are positioned on the upper surface of the upper panel adjacent an upper edge of the front panel, and wherein corresponding attachment means are positioned on the upper surface of the upper panel adjacent an upper edge of the rear panel.

7. The drape of claim 6, wherein the attachment means on the upper surface of the upper panel adjacent the upper edge of the front panel are aligned with the corresponding attachment means on the upper surface of the upper panel adjacent an upper edge of the rear panel when the drape is folded inwardly along one or more fold lines to maintain the sterility of the upper panel prior to use of the drape in a surgical procedure.

8. The drape of claim 1, wherein the upper panel includes one or more pleats, wherein the pleats are expandable outwardly when the drape is in use to surround the portion of the imaging device.

9. The drape of claim 1, wherein at least one handle is present on the rear panel.

10. The drape of claim 1, wherein a support structure is attached at an upper edge of the rear panel, a lower edge of the rear panel, or a combination thereof.

11. The drape of claim 1, wherein an attachment means is disposed on the lower surface of the upper panel adjacent the front panel.

12. The drape of claim 1, wherein the front panel is positioned a distance away from a front edge of the upper panel toward the rear panel.

13. The drape of claim 1, wherein the drape is free of sidewalls positioned between the front panel and the rear panel.

14. The drape of claim 1, wherein a first side edge of the rear panel and a first side edge of the front panel define the opening, further wherein a sidewall is positioned between a second side edge of the rear panel and a second side edge of the front panel.

15. The drape of claim 1, wherein the upper panel, the front panel, the rear panel, or a combination thereof is translucent, transparent, or radiolucent.

16. The drape of claim 1, wherein the upper panel, the front panel, the rear panel, or a combination thereof is formed from a nonwoven material.

17. The drape of claim 16, wherein the nonwoven material comprises a spunbond material, a meltblown material, or a combination thereof.

18. The drape of claim 1, wherein the upper panel, the front panel, the rear panel, or a combination thereof is formed from a film.

19. The drape of claim 18, wherein the film comprises polyethylene.

20. The drape of claim 1, wherein the drape is sterilizable.
